(19) United States
(12) Patent Application Publication
(10) Pub. No.: US 2016/0338578 A1
(43) Pub. Date: Nov. 24, 2016

(54) METHOD AND APPARATUS FOR ACQUISITION OF VOLUMETRIC IMAGING DATA WITHIN AN ANATOMIC STRUCTURE

(71) Applicant: THE GENERAL HOSPITAL CORPORATION, Boston, MA (US)

(72) Inventors: GUILLERMO J. TEARNEY, Cambridge, MA (US); TIMOTHY N. FORD, Allston, MA (US); ROBERT W. CARRUTH, Arlington, MA (US); KENGYEH CHU, Jamaica Plains, MA (US)

(21) Appl. No.: 15/111,951

(22) PCT Filed: Jan. 15, 2015
(86) PCT No.: PCT/US15/11512

§ 371 Date: Jul. 15, 2016

Related U.S. Application Data
(60) Provisional application No. 61/928,870, filed on Jan. 17, 2014.

(51) Int. Cl. A61B 1/04; A61B 1/00

(52) U.S. Cl. CPC A61B 1/041; A61B 1/00016; A61B 1/00128; A61B 1/00158; A61B 1/0016

(57) ABSTRACT

Methods, apparatus and devices for providing images based on the optical phase gradients within a biological sample can be provided. In one exemplary embodiment, a tethered and/or untethered capsule can be introduced into a biological specimen to acquire images by illuminating the specimen from at least two different angles, while an objective lens arrangement can relay an image from the illuminated sample to an imaging sensor array. The difference between images acquired using differing illumination angles and from the same position in the sample can indicate a phase contrast in a certain technique (e.g., oblique back illumination). The image focal plane provided by this exemplary arrangement/device, e.g., being planar by definition, constitutes only a small fraction of a volumetric specimen. To provide, e.g., three-dimensional or volumetric imaging, according to one exemplary embodiment of the present disclosure, it is possible to utilize a translational and rotational drive mechanism by which the thin plane of imaging is swept through the sample such that a large volume is imaged over time. To improve the coverage of the image plane sweep, according to yet another exemplary embodiment of present disclosure, it is possible to utilize a tilted camera and/or a non-paraxial optical lens arrangement, such that the image focal plane is not perpendicular to the optical axis of the objective lens arrangement.

### CROSS REFERENCE TO RELATED APPLICATION(S)

The present application relates to and claims priority from U.S. Provisional Patent Application Ser. No. 61/928,870 filed Jan. 17, 2014, the disclosure of which is incorporated herein by reference in its entirety.

### FIELD OF THE DISCLOSURE

The present disclosure relates to exemplary embodiments of method, device and apparatus for an acquisition of volumetric imaging data within an anatomic structure.

### BACKGROUND INFORMATION

Optical imaging in the field of diagnostic medicine can be limited by the mismatch between planar image data acquired by standard optical imaging systems and the non-planar, three-dimensional form of anatomical structures. While some tissues in the body are approximately planar and may be suitable for investigation by planar imaging instruments (e.g., squamous epithelium in smooth-walled luminal structures), many biological structures are inherently three-dimensional and require imaging systems with three-dimensional capabilities. These non-planar anatomical structures can include thick multi-layered tissue found in the skin, gastrointestinal tract, and cardiopulmonary vessels; and highly vascular tissues with vessels and ducts connecting superficial and deep aspects. Even tissues with planar qualities on a microscopic scale can present with complex topological features on a macro scale (e.g., folds found in gastrointestinal tissues) necessitating continuous refocusing while translating the field of view over the entire organ.

Traditional techniques for acquiring three-dimensional image information require physically moving optical elements to shift the image plane in the axial direction (i.e. tissue depth-scanning). In this regime, three-dimensional scanning over a wide coverage area generally requires scanning in both lateral and axial directions, greatly increasing the imaging time and system complexity. Increased scan time is associated with an increased presence of motion artifacts and lower patient compliance. Additionally, system complexity can increase manufacturing and maintenance costs and limits physical miniaturization.

Thus, there may be a need and benefit to provide methods, systems and apparatus for the acquisition of volumetric imaging data within an anatomic structure, which can overcome at least some of the above-described issues and/or deficiencies.

### SUMMARY OF EXEMPLARY EMBODIMENTS

These and other similar objects can be achieved with exemplary methods and apparatus for the acquisition of volumetric imaging data within an anatomic structure.

According a first exemplary embodiment of the present disclosure, apparatus and method can be provided for obtaining image information for at least one portion of at least one anatomical structure. For example, at least one housing can be provided in the anatomical structure(s). With at least one detector first arrangement provided in the housing(s), it is possible to receive planar image data regarding the at least one portion therefrom. Further, using at least one translation-causing second arrangement provided in the at least one housing, it is possible to (i) rotate and/or spin the first arrangement(s) within the at least one anatomical structure, and (ii) change an image plane of the first arrangement.

According a second exemplary embodiment of the present disclosure, further apparatus and method can be provided for obtaining image information for at least one portion of at least one anatomical structure. For example, at least one housing can be provided in the anatomical structure(s). With at least one detector first arrangement provided in the housing(s), it is possible to receive linear image data regarding the at least one portion therefrom. Further, using at least one translation-causing second arrangement provided in the at least one housing, it is possible to (i) rotate and/or spin the first arrangement(s) within the at least one anatomical structure, and (ii) change an image line of the first arrangement.

In addition, the first arrangement(s) can be further configured to obtain multiples of the planar image data during the spatial translation of the portion(s) so as to generate a volumetric image of thereof. At least one computer arrangement can be provided which can be configured to generate at least one volumetric image of the portion(s) based on the planar data as being obtained as a function of at least one of a location or an orientation of the image plane of the first arrangement(s). The computer arrangement can be provided within the housing and/or outside thereof. The computer arrangement can be configured and/or programmed to control a transmission of different radiations to the portion(s) to be provided at different sections thereof. The first arrangement(s) can receive return radiation from the portion that(s) are based on the different radiations to generate further data, and determine a phase of the portion(s) based on the further data. The different and return radiations can be electromagnetic radiations having at least one vacuum wavelength in a visible range(e.g., 400 nm to 700 nm) and/or in a near infra-red range(e.g., 700 nm to 1500 nm).

According to a further exemplary embodiment of the present disclosure, the image plane can be controlled to be non-parallel to a plane of extension of a surface of the portion(s). The change of the image plane can be automatic. The housing can be a capsule insertable into the portion(s). In one exemplary embodiment, the capsule can be tethered via a tether and/or tether-less, and the second arrangement can include a torque-communicating coil provided within the tether and/or an electric motor provided within the housing. Alternatively or in addition, the second arrangement can include a device configured to generate a magnetic field outside the anatomical structure(s). It is possible to provide an electrical power-providing device that is situated within the housing and powering the first arrangement.

In still another exemplary embodiment of the present disclosure, a computer arrangement can be provided configured to convert the planar image data to a wirelessly-transmitted data stream. A wireless transmitter can be provided that is configured to provide wireless communication as an analog or digital radiofrequency transmission with carrier frequency in a range of about 100 MHz to 10 GHz. The wireless transmitter can also be configured to provide a wireless communication that is direct electrical conduction of a time-modulated surface electrode potential.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments of the present disclosure, in which:

FIG. 1 is a cross-sectional view of a tethered capsule device that can utilize a tilted camera array detector, according to a first exemplary embodiment of the present disclosure, which can be oriented such that it is not perpendicular to the optical axis of the objective lens arrangement, with separate light sources for generating oblique back-illumination images;

FIG. 2 is a cross-section view of a tethered capsule device, according to a second exemplary embodiment of the present disclosure;

FIG. 3 illustrates a third exemplary embodiment of a capsule device that utilizes a tilted camera line detector according to the present disclosure, which is oriented such that it is not perpendicular to the optical axis of the objective lens arrangement;

FIG. 4 is a cross-section view of an untethered capsule device that utilizes a tilted camera array detector, according to a fourth exemplary embodiment of the present disclosure, which is oriented such that it is not perpendicular to the optical axis of the objective lens arrangement;

FIG. 5 is an illustration of an exemplary utilization of the tethered capsule device according to the exemplary embodiments of the present disclosure introduced into the esophagus of a patient. Electrical power and electrical signals are passed through the tether and rotary junction, which maintains power and signal contact throughout a rotation of the capsule imaging components;

FIG. 6 is an illustration of an exemplary utilization of the untethered capsule device according to the exemplary embodiments of the present disclosure introduced into the esophagus of a patient;

FIG. 7 is a flow diagram of a first exemplary procedure for an exemplary operation of the exemplary embodiments of the capsule device to obtain volumetric phase gradient images from a biological sample using a tethered capsule, according to an exemplary embodiment of the present disclosure; and

FIG. 8 is a flow diagram of a second exemplary procedure for an exemplary operation of the exemplary embodiments of the capsule device to obtain volumetric phase gradient images from a biological sample using an untethered capsule.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described exemplary embodiments without departing from the true scope and spirit of the subject disclosure, as defined by the appended claims.

### DETAILED DESCRIPTION OF EXEMPLARY EMBODIMENTS

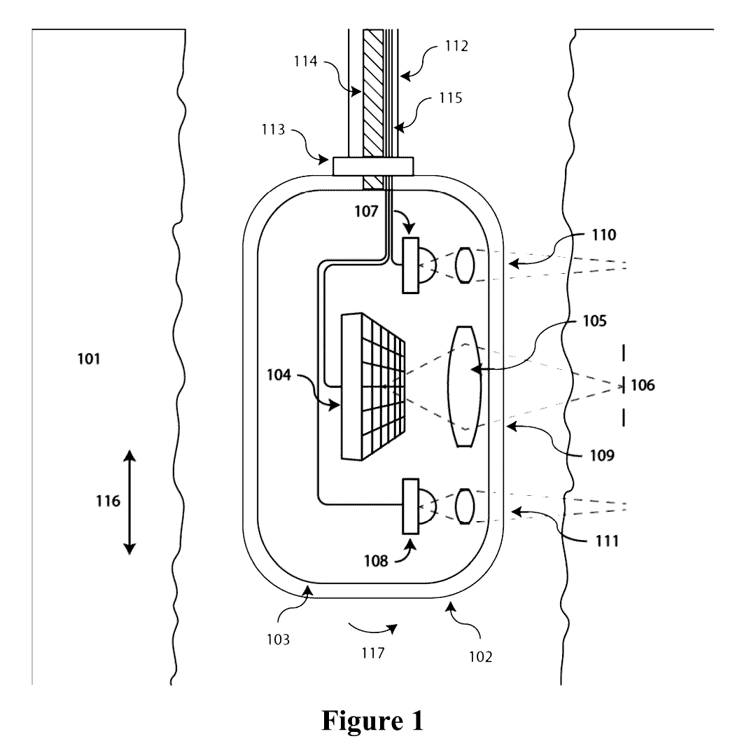

FIG. 1 illustrates a cross-sectional view of a tethered imaging capsule device that can utilize a tilted camera array detector according to an exemplary embodiment of the present disclosure. The exemplary imaging capsule device of FIG. 1 can include illumination sources (107, 108), imaging sensors (104), and optics (105) that can provide oblique back-illumination microscopy of the biological sample into which the capsule is introduced. The individual illuminations (107, 108) are separately conducted into the sample (101), and not necessarily aligned directly to the image plane (106). The light or other electro-magnetic radiation from each illumination (110, 111) can be scattered within the volume of the sample, a fraction of which can traverse the image plane from the side farther from the capsule. The selection of individual light sources can have an effect on the illumination angle of light crossing the imaging plane. A phase gradient in the plane of focus can bend the illumination light (or other electro-magnetic radiation). Depending upon the magnitude and direction of such phase gradients, using such exemplary device, it is possible to increase or decrease the amount of light (or other electro-magnetic radiation) relayed to the imaging sensor (104) by the optical lens due to the oblique nature of the original illumination. The difference between images acquired using alternating illumination direction indicates the effect of angle changes induced by phase gradients, providing a contrast mechanism for revealing phase gradients simply by contrasting the different images resulting from each illumination direction. Furthermore, multiple (e.g., three or more) images resulting from multiple illumination directions can provide phase gradient vector information from each pixel location in the sample.

To provide volumetric imaging of biological samples that can be approximately cylindrical, such as the esophagus or the intestine, additional techniques and/or components can be used, since oblique the illumination microscopy as described in herein can provide a single plane of imaging. By tilting the imaging detector (104), instead of mounting it perpendicularly to the optical imaging axis, the imaging focal plane (106) would also be tilted, i.e., being non-perpendicular to the optical imaging axis. Thus, the focal plane (106) can include, e.g., line segments found at different distances from the lens, e.g., at different depths within the sample (101). Further, by a rotation (117) of the exemplary capsule (102) relative to the sample (101), and/or by rotating the contents of the capsule (102) within the respective housing (103), each such line (e.g., each being at a different depth in the sample) can trace a cylinder of different radius. In an image sequence acquired by the array of the detector (104) during a rotation of the capsule optics, each pixel can trace a circle in the imaging sample. For example, one spatial coordinate of the sensor array can encode depth, and the other can encode longitudinal position relative to the capsule. The capsule can then be further translated in the longitudinal direction (116), either driven by the tether or by natural forces such as gravity or peristaltic action of an organ such as the esophagus, in order to image a greater extent of the sample.

Alternatively, with another embodiment of the present disclosure, it is possible to utilize modes of motion other than rotation and translation. Any direction of motion of the focal plane, and in any sequence, that volumetrically samples the biological specimen, can be used to form volumetric imaging data.

According to one exemplary embodiment of the present disclosure, illumination sources (107, 108), imaging sensors (104), and optics (105) can be contained within the capsule device (102). In a further exemplary embodiment, a tether (112) can connect the capsule device (102) with an external electrical and computer system outside of the imaging sample (101) or patient. The tether (112) can contain electrical wiring (115) that can provide power to the components within the capsule device (102), and can conduct and/or provide the image data in the form of, e.g., electrical signals from the sensor array to the external system. The tether (112) can also contain a driveshaft (114), which can be a mechanical component that can transmit torque applied by a motor at one end of the tether to the capsule or its contents, facilitating the optical system to rotate, as described herein. A rotary junction can be provided at or in the tether (112) that can facilitate an electrical contact for power and signal(s) to and from the capsule device (102) to be preserved while the driveshaft (114) is rotated. Alternatively or in addition, a motor can also be positioned within the capsule device (102), e.g., so as to drive the rotation directly and drawing electrical power from the wiring in the tether (112). According to another exemplary embodiment of the present disclosure, it is possible to facilitate a rotation and/or a translation of the capsule device (102) by modifying and/or manipulating an external magnetic field. For example, the external system can receive the imaging data from the capsule sensor, and can process such data into phase gradient volumetric images.

In yet other exemplary embodiments of the present disclosure, the imaging modality may be other than an oblique back-illumination microscopy. For example, volumetric imaging can be performed using any technique that can generate a planar focal plane, including but not limited to bright field microscopy, reflectance microscopy, reflectance confocal microscopy, fluorescence microscopy, multiple-wavelength reflectance microscopy, spectrally encoded confocal microscopy, multiple-wavelength excitation fluorescence microscopy, Fourier microscopy, or coherence microscopy, including full field optical coherence tomography (FFOCT) and full field optical coherence microscopy (FFOCM).

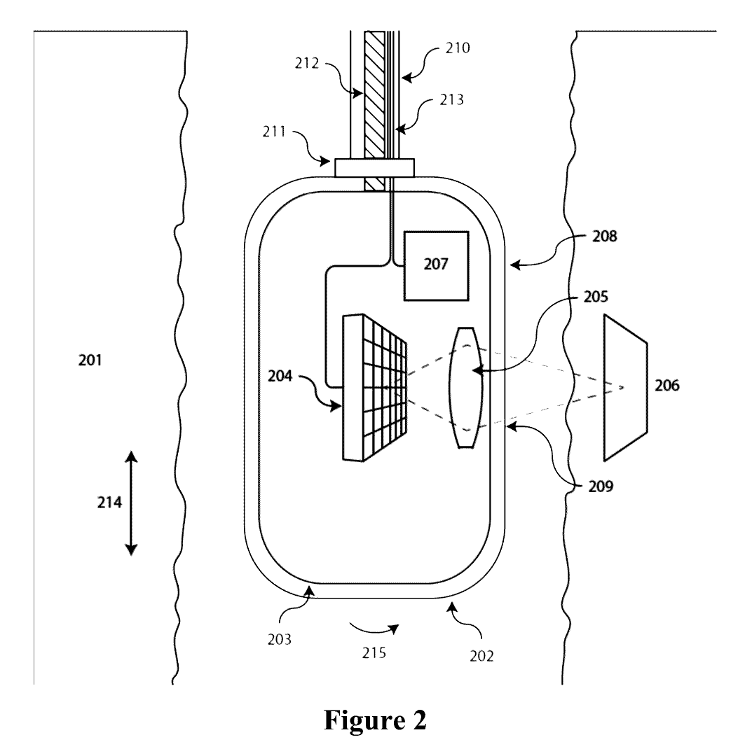

FIG. 2 shows a cross-section view of a tethered capsule device, according to a second exemplary embodiment of the present disclosure. Using this exemplary tether capsule device (202), the image plane (206) can be tilted such that it is not perpendicular to the optical axis of the objective lens arrangement (205), as suitable for microscopy techniques such as bright field microscopy, reflectance microscopy, reflectance confocal microscopy, fluorescence microscopy, multiple-wavelength reflectance microscopy, spectrally encoded confocal microscopy, multiple-wavelength excitation fluorescence microscopy, Fourier microscopy, or coherence microscopy, including full field optical coherence tomography (FFOCT) and full field optical coherence microscopy (FFOCM). For example, using such exemplary embodiment, it is possible to implement the techniques described herein above by acquiring different images while tilting the planar focal plane (206), following illumination by different light (or other radiation) sources (207), and/or by moving a component within the apparatus mechanically, including changing the path length of a reference arm for a coherence embodiment such as FFOCT and FFOCM. The same components shown in FIG. 1 are shown in FIG. 2, with the reference increase by 100 (i.e., 202, 204, etc.).

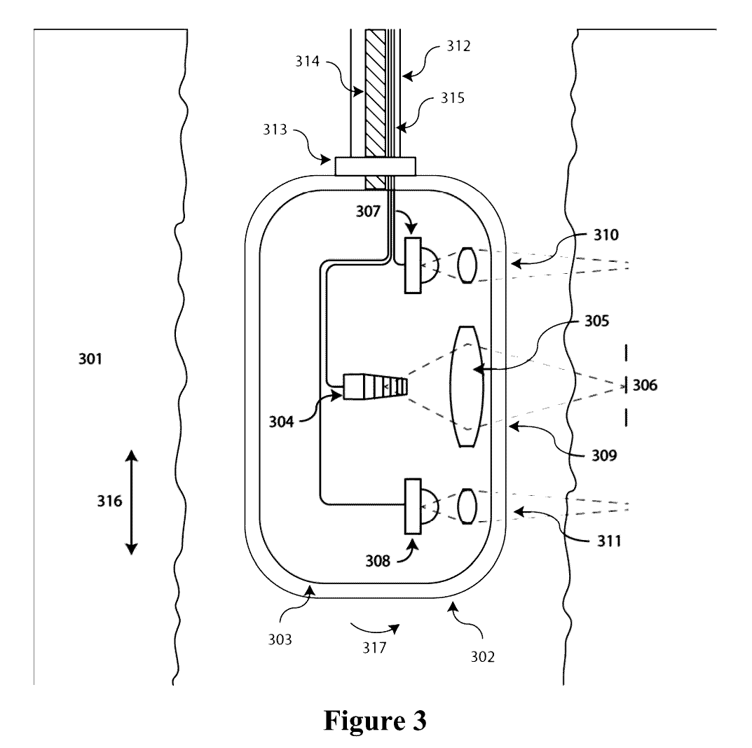

In an alternative exemplary embodiment of the capsule device (302) according to the present disclosure, as illustrated in FIG. 3, the imaging sensor may be and/or include a linear array (304), e.g., instead of an area array. For example, tilting this array (304) relative to the optical axis of the imaging optics (305) can cause each pixel along this line to be focused to a different depth in the sample (306), and the capsule device (302) or its contents (303) can be rotated (317) such that each sensor pixel can trace a circle at different depth. The exemplary operating principle of the exemplary embodiment shown in FIG. 3 is similar to that of the exemplary embodiment of the capsule device shown in FIG. 1, with an exemplary difference being a lack of additional pixels along the longitudinal axis results in a more limited imaging volume, preferring additional rotations as the capsule device (302) translates to form a similar volume of imaging. The same components shown in FIG. 2 are shown in FIG. 3, with the reference increase by 100 (i.e., 302, 304, etc.).

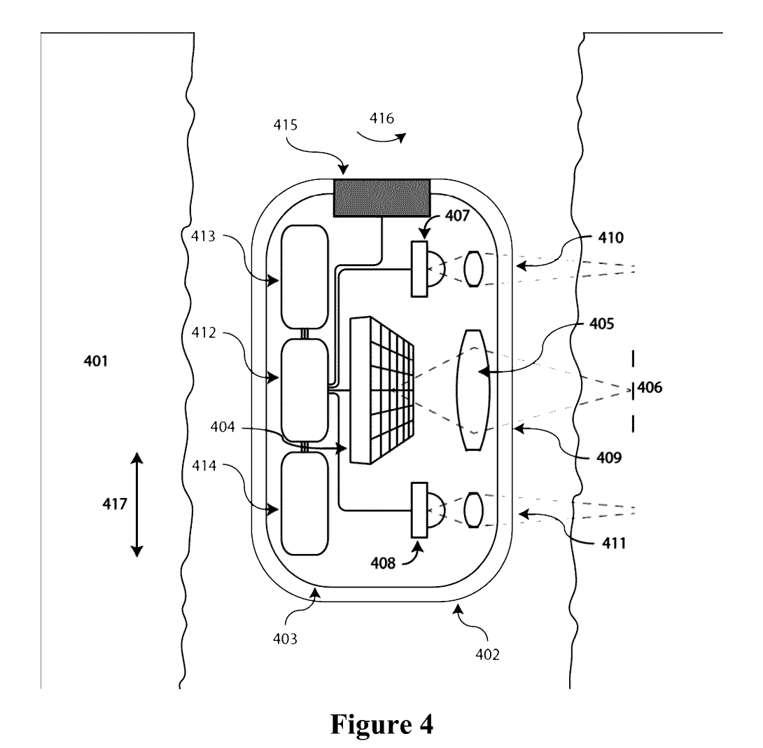

In a further alternative exemplary embodiment of the present disclosure, as shown in FIG. 4, the exemplary capsule device (402) can be self-contained and not physically tethered to the external system. In this exemplary embodiment, a power source (413) in the capsule (e.g., a battery) can provide an electrical power to the illumination/radiation sources (407, 408) and/or sensor components (404). Further, as shown in FIG. 4, a wireless mechanism (414) can be provided for a transmission of imaging data to the external system. The wireless mechanism (414) can be or include, for example, a device which provides analog and/or digital signal transmission using electromagnetic waves with radio frequencies between, e.g., about 100 MHz and 10 GHz, and/or digital signal transmission using modulated potential of surface electrodes and direction electrical conduction through the body. An on-board circuitry of the capsule device (402) can include a processor (412) to obtain data from the imaging sensor, and convert such data to a format amenable to a wireless transmitter, which can also be provided on or in the capsule device (402). The processor (412) can be specifically programmed to provide, for example, image compression and bandwidth-reducing, contrast enhancement and/or de-noising filtering. The external system, according to this exemplary embodiment, can include at least one wireless receiver (414) and/or at least one surface potential electrode, as well as any hardware and/or software used to decode the imaging data from the wireless data stream. The same components shown in FIG. 3 are shown in FIG. 4, with the reference increase by 100 (i.e., 402, 404, etc.).

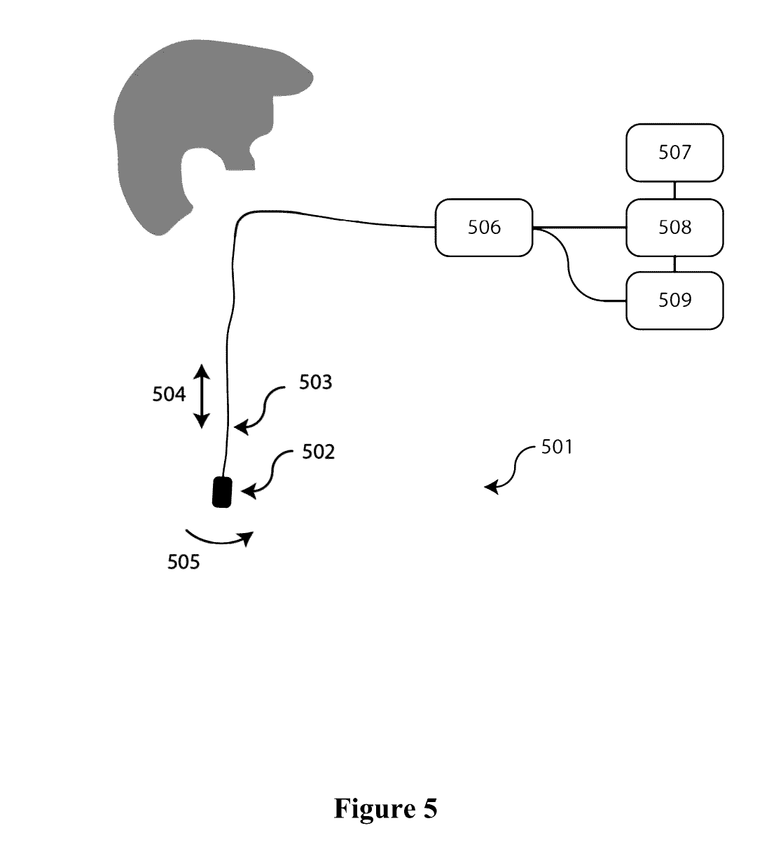

FIG. 5 illustrates an exemplary usage/application of the exemplary embodiment of the capsule device (502) in the esophagus of a patient (501). The capsule device (502) can be introduced into the esophagus. The capsule device (502) can be rotated (505), and/or translated (504) within the organ (e.g., the esophagus) to generate volumetric imaging. The flexible tether (503) can link the capsule device (502) with the external system, which can supply power through the tether (503) to the capsule device (502). A computer (508), which can be provide on or in the external system, e.g., can process the imaging data into volumetric phase images, and can include or be connect to an image display (507) and a power source (509) for the tethered capsule device (502). A rotary junction (506) can be provided which can facilitate an electrical contact between the external system and the capsule device (502) to be maintained even while the capsule or its contents are rotated.

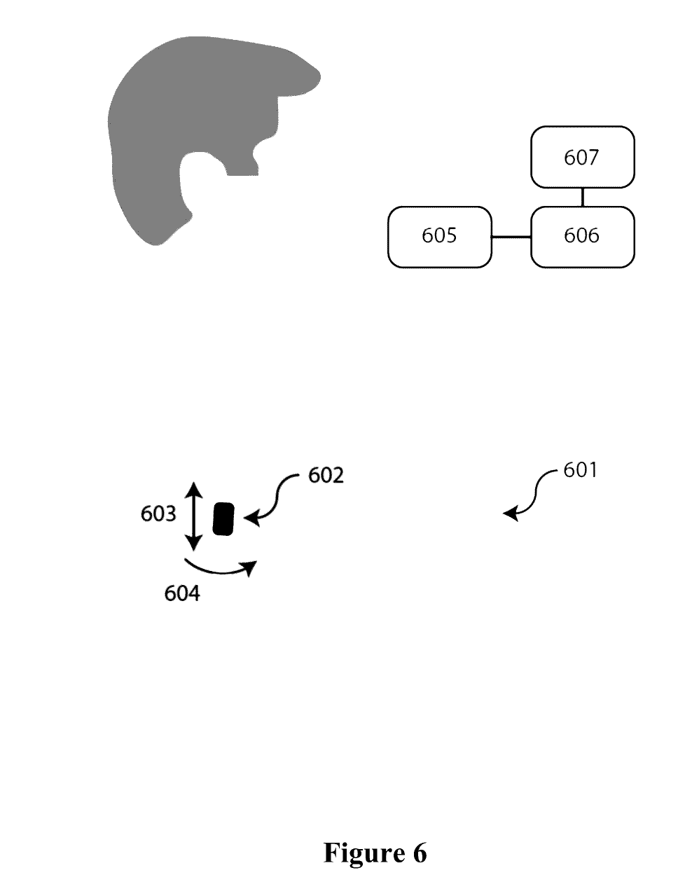

FIG. 6 illustrates an exemplary usage/application of the exemplary embodiment of the untethered capsule device 602 (the example of which is shown in FIG. 4), in which image data provided by the capsule is transmitted wirelessly to a transceiver and computer system for storage and display. For example, according to one exemplary embodiment of the present disclosure, the exemplary untethered capsule (602) and/or its contents can be rotated (604) and/or translated (603). In such exemplary configuration shown in FIG. 6, no physical link connects the capsule device (602) to the external system. Being self-contained, the capsule device (602) can be powered by an onboard battery, which can supply the electrical power for illumination, processing and control circuitry, wireless transmission, and the sensor array. Imaging data generated using this exemplary embodiment of the capsule device (602) can be transmitted wirelessly to a transceiver (605) on the external system for additional processing by a processor/computer (606), storage, and/or display on a display device (607).

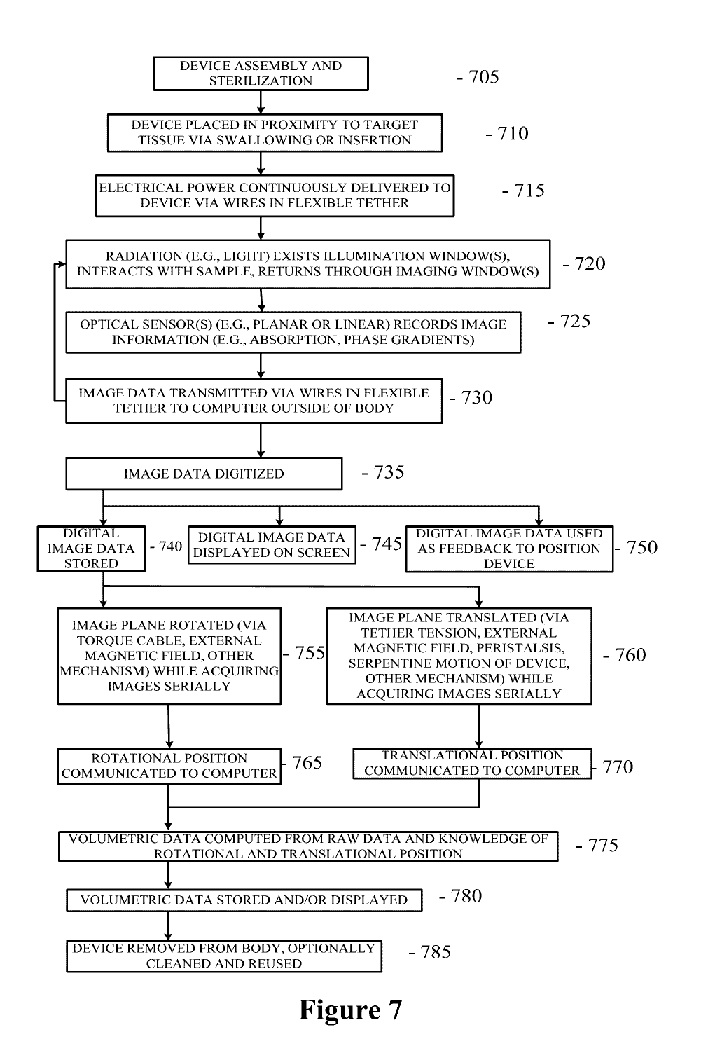

FIG. 7 shows a flow diagram that illustrates (and provides details of) exemplary utilization procedures for a tethered capsule device, according to an exemplary embodiment of the present disclosure. For example, in step 705, the exemplary capsule device is assembled and sterilized. In step 710, the exemplary device can be place in the proximity of the target tissue, e.g., by swallowing or insertion. In step 715, electrical power can be continuously delivered to the exemplary device, e.g., via wires in a flexible tether. Then, in step 720, electro-magnetic radiation (e.g., light) can be provided so as to exit the window(s) of the device, interact with the sample/tissue, and return through such window(s). Optical sensors(s) (e.g., planar and/or linear) can record image information from the target tissue (e.g., including absorption information, gradients, etc.) in step 725. In step 730, the image information (or image information) can be transmitted (e.g., via wires in the flexible tether) to a computer outside of the body, and in step 735, the computer digitizes the image information. For example, the digital image data (or information) can be stored (step 740), displayed on the screen (step 745) and/or used as feedback information to position the exemplary device (step 750).

In step 755, it is possible to use a torque cable, an external magnetic field and/or other configuration or mechanism to rotate an image plane of the irradiation from and/or to the exemplary device, e.g., while obtaining or otherwise acquiring the images (e.g., serially and/or in parallel). In step 765, the rotational position of the device can be communicated to the computer. At the same time or at another time, in step 760, it is possible to use a tether tension, an external magnetic field, peristalsis, serpentine motion of the device and/or other configuration or mechanism to translate the image plane of the irradiation from and/or to the exemplary device, e.g., while obtaining or otherwise acquiring the images (e.g., serially and/or in parallel). In step 770, the translational position of the device can be communicated to the computer.

Further, in step 775, the computer can be programmed to determine or otherwise compute volumetric data from the raw data (e.g., the image information) and/or based on or using the rotational position information and/or the translational position information. In step 780, the volumetric data can be stored, displayed, etc. Then, in step 785, the exemplary device can be removed from the body, and possibly cleaned and/or reused.

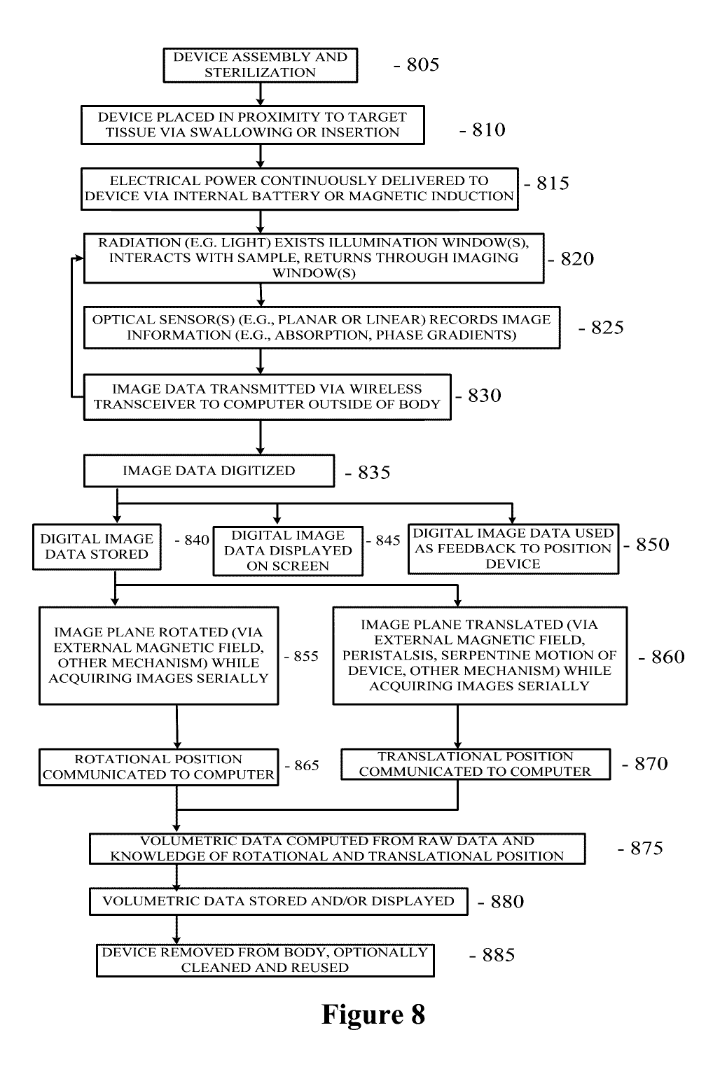

FIG. 8 shows a flow diagram that illustrates (and provides details of) exemplary utilization procedures for an untethered wireless capsule device, according to another exemplary embodiment of the present disclosure. For example, in step 805, the exemplary capsule device can be assembled and sterilized. In step 810, the exemplary device can be place in the proximity of the target tissue, e.g., by swallowing or insertion. In step 815, electrical power can be continuously delivered to the exemplary device, e.g., via an internal battery and/or magnetic induction. Then, in step 820, electro-magnetic radiation (e.g., light) can be provided so as to exit the window(s) of the device, interact with the sample/tissue, and return through such window(s). Optical sensors(s) (e.g., planar and/or linear) can record image information from the target tissue (e.g., including absorption information, gradients, etc.) in step 825. In step 830, the image information (or image information) can be transmitted (e.g., wirelessly via a wireless transceiver) to a computer outside of the body, and in step 835, the computer digitizes the image information. For example, the digital image data (or information) can be stored (step 840), displayed on the screen (step 845) and/or used as feedback information to position the exemplary device (step 850).

In step 855, it is possible to use an external magnetic field and/or other configuration or mechanism to rotate an image plane of the irradiation from and/or to the exemplary device, e.g., while obtaining or otherwise acquiring the images (e.g., serially and/or in parallel). In step 865, the rotational position of the device can be communicated to the computer. At the same time or at another time, in step 860, it is possible to use an external magnetic field, peristalsis, serpentine motion of the device and/or other configuration or mechanism to translate the image plane of the irradiation from and/or to the exemplary device, e.g., while obtaining or otherwise acquiring the images (e.g., serially and/or in parallel). In step 870, the translational position of the device can be communicated to the computer.

Further, in step 875, the computer can be programmed to determine or otherwise compute volumetric data from the raw data (e.g., the image information) and/or based on or using the rotational position information and/or the translational position information. In step 880, the volumetric data can be stored, displayed, etc. Then, in step 885, the exemplary device can be removed from the body, and possibly cleaned and/or reused.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements and methods which, although not explicitly shown or described herein, embody the principles of the disclosure and are thus within the spirit and scope of the present disclosure. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety. All publications referenced herein can be incorporated herein by reference in their entireties.

1. An apparatus for obtaining image information for at least one portion of at least one anatomical structure, comprising:
at least one housing which is configured to be provided in the at least one anatomical structure, and comprising:
at least one detector first arrangement which is configured to receive planar image data regarding the at least one portion therefrom, and
at least one translation-causing second arrangement which is configured to (i) rotate or spin the at least one first arrangement within the at least one anatomical structure, and (ii) change an image plane of the first arrangement.

2. The apparatus according to claim 1, wherein the at least one first arrangement is configured to obtain at least one of (i) a plurality of the planar image data during the rotation or spinning thereof, or (ii) multiples of the planar image data during the spatial translation of the at least one portion so as to generate a volumetric image of the at least one portion.

3. (canceled)

4. The apparatus according to claim 1, further comprising at least one computer arrangement which is configured to generate at least one volumetric image of the at least one portion based on the planar data as being obtained as a function of at least one of a location or an orientation of the image plane of the at least one first arrangement.

5. The apparatus according to claim 4, wherein the at least one computer arrangement is provided within the housing.

6. The apparatus according to claim 4, wherein the at least one computer arrangement is provided outside of the housing.

7. The apparatus according to claim 4, wherein the at least one computer arrangement controls a transmission of different radiations to the at least one portion to be provided at different sections thereof, wherein the at least one first arrangement receives return radiation from the at least one portion that are based on the different radiations to generate further data, and wherein the at least one computer arrangement is configured to determine a phase of the at least one portion based on the further data.

8. The apparatus according to claim 7, wherein the different and return radiations are electromagnetic radiations having at least one vacuum wavelength in at least one of (i) a visible range, or (ii) a near infra-red range.

9. The apparatus according to claim 8, wherein the visible range is 400 nm to 700 nm.

10. (canceled)

11. The apparatus according to claim 7, wherein the near infra-red range is 700 nm to 1500 nm.

12. The apparatus according to claim 1, wherein the image plane is controlled to be non-parallel to a plane of extension of a surface of the at least one portion.

13. The apparatus according to claim 1, wherein the change of the image plane is automatic.

14. The apparatus according to claim 1, wherein the at least one housing is a capsule insertable into the at least one portion.

15. The apparatus according to claim 14, wherein the capsule is tethered via a tether.

16. The apparatus according to claim 15, wherein the second arrangement includes at least one of (i) a torque-communicating coil provided within the tether, or (ii) an electric motor provided within the at least one housing.

17. (canceled)

18. The apparatus according to claim 14, wherein the capsule is tether-less.

19. The apparatus according to claim 18, wherein the second arrangement includes at least one of (i) an electric motor provided within the at least one housing, or (ii) a device configured to generate a magnetic field outside the at least one anatomical structure.

20. (canceled)

21. The apparatus according to claim 18, further comprising an electrical power-providing device situated within the at least one housing and powering the first arrangement.

22. The apparatus according to claim 18, further comprising a computer arrangement configured to convert the planar image data to a wirelessly-transmitted data stream.

23. The apparatus according to claim 22, further comprising a wireless transmitter configured to provide a wireless communication at least one of (a) as an analog or digital radiofrequency transmission with carrier frequency in a range of about 100 MHz to 10 GHz, or (ii) that is a direct electrical conduction of a time-modulated surface electrode potential.

24. (canceled)

25. An apparatus for obtaining image information for at least one portion of at least one anatomical structure, comprising:
at least one housing which is configured to be provided in the at least one anatomical structure, and comprising:
at least one detector first arrangement which is configured to receive linear image data regarding the at least one portion therefrom, and
at least one translation-causing second arrangement which is configured to (i) rotate or spin the at least one first arrangement within the at least one anatomical structure, and (ii) change an image line of the first arrangement.

26-48. (canceled)

49. A method for obtaining image information for at least one portion of at least one anatomical structure, comprising:
providing at least one housing in the at least one anatomical structure;
with at least one detector first arrangement provided in the at least one housing, receiving planar image data regarding the at least one portion therefrom, and
with at least one translation-causing second arrangement provided in the at least one housing, (i) rotating or spinning the at least one first arrangement within the at least one anatomical structure, and (ii) changing an image plane of the first arrangement.

50. A method for obtaining image information for at least one portion of at least one anatomical structure, comprising:
providing at least one housing in the at least one anatomical structure;
with at least one detector first arrangement provided in the at least one housing, receiving linear image data regarding the at least one portion therefrom, and
with at least one translation-causing second arrangement provided in the at least one housing, (i) rotating or spinning the at least one first arrangement within the at least one anatomical structure, and (ii) changing an image line of the first arrangement.
